(19) United States
(12) Patent Application Publication
(10) Pub. No.: US 2010/0298305 A1
(43) Pub. Date: Nov. 25, 2010

(54) TIZANIDINE FOR THE TREATMENT OF POST-TRAUMATIC STRESS DISORDER AND NIGHTMARES

(75) Inventor: Bruce Capehart, Chapel Hill, NC (US)

Correspondence Address: KLARQUIST SPARKMAN, LLP, 121 SW SALMON STREET, SUITE 1600, PORTLAND, OR, 97204, US

(73) Assignee: The United States Government, as represented by the Department of Veterans Affairs

(21) Appl. No.: 12/626,500

(22) Filed: Nov. 25, 2009

Related U.S. Application Data
(60) Provisional application No. 61/118,353, filed on Nov. 26, 2008.

(51) Int. Cl. A61K 31/551; A61K 31/433; A61P 25/16; A61P 25/00; A61P 25/24; A61P 25/18; A61P 25/08; A61P 25/26; A61K 31/506; A61K 31/454

(52) U.S. Cl. 514/221; 514/362; 514/252.15; 514/322

(57) ABSTRACT

Disclosed herein are methods of treating a psychiatric disorder, such as a sleep disorder, an anxiety disorder, a mood disorder or a perceptual disturbance. In one example, a method of treating a sleep disorder or anxiety disorder includes selecting a subject with a sleep disturbance or anxiety disorder in the absence of an underlying physical disorder and administering a therapeutically effective amount of tizanidine, thereby reducing or inhibiting a symptom of the sleep disorder or anxiety disorder. In another example, a method of treating a mood disorder or a perceptual disturbance is disclosed. This method includes selecting a subject with a mood disorder or perceptual disturbance and administering a therapeutically effective amount of tizanidine, thereby reducing or inhibiting a symptom of the mood disorder or perceptual disturbance.

### CROSS-REFERENCE TO RELATED APPLICATION

This application claims the benefit of U.S. Provisional Application No. 61/118,353 filed Nov. 26, 2008, which is herein incorporated by reference in its entirety.

### FIELD OF THE DISCLOSURE

This disclosure relates to the field of psychiatric disorders and in particular, to methods for treating disorders such as post traumatic stress syndrome, with a α2-adrenergic agonist or imidazoline agonist.

### BACKGROUND

There is an ongoing need for effective pharmacologic treatment for psychiatric disorders. The signs and symptoms of psychiatric disorders continue to exert significant morbidity upon persons suffering from them. Inadequately treated psychiatric disorders remain a serious risk factor for suicide and/or violence toward other persons and negatively affect quality of life for the patient and their family members. While there are pharmacologic treatments for psychiatric disorders available today, some unmet needs still exist. Several of these unmet treatment needs include: nightmares and other symptoms associated with Post-Traumatic Stress Disorder (PTSD); reducing Panic Disorder signs and symptoms (such as subjective anxiety, agoraphobia and panic attacks); decreasing psychotic symptoms (such as hallucinations or delusions); improving attention and concentration (such as when the impairment results from a condition other than Attention-Deficit Hyperactivity Disorder); and improving mood.

Psychiatric diagnoses are most commonly determined by comparing a subject's presenting complaint(s) to a standardized diagnostic manual such as the American Psychiatric Association's Diagnostic and Statistical Manual, Fourth Edition (DSM-IV; American Psychiatric Publishing, Inc., Washington D.C., 2000). The DSM-IV uses a symptom-based diagnostic system because it is commonly recognized by persons skilled in the art of diagnosis and treatment of psychiatric conditions that several different underlying causes could result in a similar clinical presentation. For example, the group of mood symptoms recognized by lay persons as “depression” would be known to those skilled in the art of diagnosis and management of psychiatric disorders as possibly resulting from major depression, biopolar disorder, use/abuse of one of any number of substances (e.g., alcohol, cocaine), a neurological disorder (e.g., traumatic brain injury), an untreated or under-treated anxiety disorder (e.g., panic disorder, obsessive-compulsive disorder, PTSD), or a psychotic disorder (e.g., schizophrenia). However, psychiatric medications are commonly prescribed based upon the presenting complaint(s) because specific medications can be useful in reducing psychiatric signs or symptoms even if the underlying cause is not clear.

### SUMMARY

Disclosed herein are methods of treating a psychiatric disorder, such as a sleep disorder, an anxiety disorder, a mood disorder or a perceptual disturbance. In one example, a method of treating a sleep disorder, anxiety disorder, mood disorder or perceptual disturbance includes selecting a subject with the disorder in the absence of an underlying physical disorder and administering a therapeutically effective amount of tizanidine, thereby reducing or inhibiting a symptom of the sleep disorder or anxiety disorder. In another example, a method of treating a mood disorder or a perceptual disturbance is disclosed. This method includes selecting a subject with a mood disorder or perceptual disturbance and administering a therapeutically effective amount of tizanidine, thereby reducing or inhibiting a symptom of the mood disorder or perceptual disturbance. In some examples, the method of treating a sleep disorder, anxiety disorder, mood disorder or perceptual disturbance further includes administering a therapeutically effective amount of an additional therapeutic compound such as an antidepressant, antipsychotic, mood stabilizer, anticonvulsant, benzodiazepine, or a combination thereof. In other examples, the method includes administering a therapeutically effective amount of tizanidine in the absence of a stimulatory agent, such as a central nervous system stimulatory agent.

The foregoing and other feature of the disclosure will become more apparent from the following detailed description.

### DETAILED DESCRIPTION

### I. Abbreviations and Terms

BDI-II: Beck depression inventory-II

BVMT-R: brief visuospatial memory test-revised

CLVT-II: California verbal learning test-II

DTS: Davidson trauma scale

DSM-IV: diagnostic and statistical manual, fourth edition

MMPI-2: Minnesota multiphasic personality inventory-2

MOS: military occupational specialty

MRI: magnetic resonance imaging

OCD: obsessive-compulsive disorder

PMDD: premenstrual dysphoric disorder

PTSD: post-traumatic stress disorder

RCFT: Rey-Osterrieth complex figure test

SSRI: selective serotonin reuptake inhibitor

TBI: traumatic brain injury

WAIS-III: Wechsler adult intelligence scale-III

WCST: Wisconsin card sorting test

WTAR: Wechsler test of adult reading

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

Absent an underlying physical disorder: A phrase used to describe a disorder that is already known to be reduced or prevented by treatment with a centrally acting α2 adrenergic agonist, such as tizanidine. For example, the method includes selecting a subject that does not have a disorder or condition that includes spasms, cramping, and tightness of muscles caused by medical problems such as multiple sclerosis, spastic diplegia, back pain, or certain other injuries to the spine or central nervous system that could be relieved or prevented by treatment with a centrally acting α2 adrenergic agonist (e.g., tizanidine). In other examples, the method includes selecting a subject that does not have a traumatic brain injury.

Administration: To provide or give a subject an agent, such as tizanidine, by any effective route. Exemplary routes of administration include, but are not limited to, oral; injection, continuous or intermittent infusion (such as subcutaneous, intramuscular, intradermal, intrathecal, epidural, intracranial, intraperitoneal, and intravenous); sublingual; rectal; transdermal; intranasal; vaginal; and inhalation routes.

Alpha-2 adrenergic agonists: A compound that selectively stimulates α2-adrenergic receptors. Exemplary α2 agonists include dexmedetomidine, clonidine, lofexidine, moxonidine, xylazine, tizanidine and guanfacine.

Anxiety disorder: Any disorder characterized by increased anxiety. Some common anxiety disorders are social phobia, post-traumatic stress disorder, panic disorder, panic attacks, and obsessive-compulsive disorder. Anxiety disorders can be accompanied by perceptual disturbances. Anxiety disorders can be caused by a medical disorder (e.g., endocrine disorders, lupus), medication side-effect (e.g., interferon), substance use disorder, neurologic disorder (e.g., seizure disorder, brain injury), or have no clear cause.

Brain injury: Any injury to one of more types of intra-cranial tissues, regardless of injury mechanism. While the most common injury mechanisms are blunt or penetrating trauma, other injuries may include surgical trauma, radiation, electricity/lightning, acoustic energy, or a blast wave injury in its primary, secondary, tertiary, or quaternary forms. Brain injury includes the injury mechanism and any post-injury sequelae, such as the physical, biochemical, inflammatory, or immunologic responses to injury, whether such sequelae occur as an immediate, delayed, or prolonged response. Some injury mechanisms, injury responses, and post-injury clinical syndromes are discussed in C G Goetz (editor), Textbook of Clinical Neurology, 3^{rd} Edition, 2007, Chapter 51. Additional mechanisms or responses include, but are not limited to cavitation, shear, strain, or strain rate mediated injuries; blood-brain barrier response; acute or chronic inflammatory responses; and altered neurogenesis, neuronal migration, or stem cell response. Additional post-injury clinical syndromes could include changes in mood, anxiety, cognition, perception, consciousness, or reality testing.

Depressive Disorder: A mood disorder characterized by a predominantly sad or depressed mood, typically but not always of two or more weeks' duration. A depressive disorder also has other signs or symptoms accompanying a sad or depressed mood, including one or more of: decreased energy, appetite changes, weight gain or loss, insomnia or hypersomnia, recurrent thoughts or death, thoughts of suicide, loss of interest in usual activities, slowed thinking or cognitive speed, increased speech latency, decreased volume of speech, excessive or inappropriate guilt, diminished concentration, feeling sluggish, and slower than normal motor activity (such as gross motor, fine motor, speech). Depressive disorders can be accompanied by perceptual disturbances. Depressive disorders can be caused by a medical disorder (e.g., endocrine disorders, lupus), medication side-effect (e.g., interferon), substance use disorder, neurologic disorder (e.g., seizure disorder, traumatic brain injury), or have no clear cause.

Hallucination: Altered, misperceived, or incorrect sensory experiences. See “perceptual disturbances” below for additional information.

Imidazoline Receptor: A class of receptors located on a variety of cells and are activated or deactivated by specific agonists or antagonists.

Mood: A person's subjective report on their emotional perspective on self, situation, future, or past. While mood can fluctuate from states such as “happy,” “sad,” “angry,” or “pleased” within a day, a prolonged state of sad or depressed mood is a defining characteristic of a depressive illness.

Mood disorder: A medical, neurologic, or psychiatric disorder with the primary sign or symptom as an alteration in mood. Mood disorders are usually classified as depressive (e.g., principal mood symptom is a sustained sad or depressed mood) or manic (e.g., principal mood symptom is a sustained expansive, elevated, or irritable mood). Symptoms or signs beyond the mood state proper may be required to diagnose a mood disorder.

Neurological disorder: Any disorder or illness of the brain, spinal cord, or peripheral nerves caused by a direct effect and/or a response to one or more mechanism or etiology. A non-limiting list of mechanisms or etiologies leading to neurological disorders includes: trauma, inflammation, injury, autoimmune, neoplastic or paraneoplastic, vascular, infection, hematologic, hemodynamic, intracellular storage defect, structural, degenerative or demyelinating, thromboembolic, renal, hepatic, developmental or malformation, gastrointestinal, body temperature, malnutrition, endogenous or exogenous metabolic derangement, genetic, endocrinologic, toxic (to include prescription medication side-effect, over-the-counter medication side-effect, effect of radiation, or illicit drug and alcohol use), necrosis, autophagy, or apoptosis.

Nightmare: A frightening dream that causes the interruption of sleep. Repeated instances of nightmares can lead to a specific sleep disorder diagnosis of Nightmare Disorder. Nightmares are also commonly observed as a symptom in PTSD and other anxiety conditions. See also “Sleep Terror” below.

Parkinson's disease: A disease of unclear etiology characterized by neuronal loss, depigmentation, presence of Lewy Bodies, and the loss of dopaminergic activity in the substantia nigra. As used herein, Parkinson's disease also refers to a disease or condition with the characteristic signs of hypokinetic movement typically associated with Parkinson's disease (e.g., side-effect of certain antipsychotic medications; trauma or cerebrovascular disease affecting the substantia nigra).

Partial complex seizure disorder: A disorder that begins with a small focal seizure that is accompanied by altered consciousness with subsequent amnesia for the event. The partial-complex seizure can be accompanied by unusual behaviors ranging from simple repeated motor activity (e.g., lip-smacking) to more complex behaviors.

Perceptual disturbance: An altered perception or conscious experience of sensory information. A common perceptual disturbance is a hallucination (incorrect perception of auditory, visual, tactile, olfactory, or gustatory sense information). Another common perceptual disturbance is a flashback (the sensory experience of being in a different place and/or time, often in response to a sensory trigger (e.g., after hearing a car backfire, a combat veteran has a momentary sensation of being back at war)). Altered reality testing is sometimes used to describe a person experiencing perceptual disturbances because the person is not accurately perceiving sensory stimuli.

Post-Traumatic Stress Syndrome (PTSD): A disorder that can occur after experiencing a traumatic event that leaves a subject feeling scared, confused, and/or angry to the extent that daily activities are difficult to perform. A traumatic event can include combat or military exposure, child sexual or physical abuse, terrorist attacks, sexual or physical assault, serious accidents, and natural disasters (such as a fire, tornado, hurricane, flood, or earthquake). In an example, PTSD is defined by the Diagnostic and Statistical Manual (DSM), Fourth-Edition, Text Revision, published by the American Psychiatric Associating (DSM-IV-TR).

Psychiatric disorder: Any disorder that results in altered or abnormal behavior, function, or subjective distress in one or more of the following intrapersonal or interpersonal realms: mood, anxiety, memory, cognition, consciousness, perception, sexual experience, sleep, substance use/addiction, personality, attention/concentration, psychosis, identity, eating, or bodily function or integrity. A psychiatric disorder typically causes the patient or others around the patient noticing social, interpersonal, and/or occupational distress or dysfunction. The cause (etiology) of a psychiatric disorder may be idiopathic (unknown), or it may be due to a recognized psychosocial stressor, a medical disorder, or a neurological disorder. In one example, a psychiatric disorder is reduced or prevented by administering a therapeutic amount of tizanidine in a subject in need of treatment. In some examples, a subject is selected that does not have an underlying physical disorder.

Seizure Disorder: A “paroxysmal event due to abnormal, excessive, hypersynchronous discharges from an aggregate of central nervous system (CNS) neurons” that may or may not result in observable changes in behavior (Chapter 363 of Harrison's Principles of Internal Medicine (Fauci A S, Kasper D L, et al. (editors), 17^{th} Edition, 2008). A seizure is a single event while epilepsy or seizure disorder is a medical diagnosis to describe a condition characterized by repeated seizures. Various types of seizures include simple partial, complex partial, partial with secondary generalization, absence, atypical absence, generalized tonic-clonic, atonic, myoclonic, or unclassified. Brain injury as defined above is a recognized cause of seizures. Seizures can be associated with various additional clinical problems: cognitive changes, mood or anxiety changes, interictal behavior changes, sudden death, psychosocial impairments, occupational problems, or psychosis.

Selective Serotonin Reuptake Inhibitor (SSRI): A type of antidepressant medication that is prescribed for the treatment of various psychiatric conditions, including, but not limited to, a depressive disorder or an anxiety disorder. Commonly prescribed SSRIs include fluoxetine, paroxetine, sertraline, citalopram, escitalopram, and fluvoxamine. Other non-limiting examples of SSRI include prodrug or pharmacologically active metabolite of these SSRI medications.

Sertraline: A selective serotonin reuptake inhibitor that is prescribed to treat one or more of the following indications: major depression or a depressive disorder, OCD, PTSD, panic disorder, social phobia, PMDD, or an anxiety disorder.

Sign: An observation, result, finding, or outcome on a medical test or examination that may indicate the presence of an associated medical, neurologic, or psychiatric condition. Non-limiting examples include observed behavior reported by a non-medical observer (e.g., family member, friend, law enforcement officer, clergy, fellow member of a military unit); observed behaviors during clinical evaluation such as anxiety noted on mental status examination; psychological or neuropsychological test results; laboratory value from blood, urine, cerebrospinal fluid; radiologic examinations such as x-rays, CT or MR scans; physical examination results such as impaired coordination or disconjugate eye movements on neurological examination, or elevated blood pressure on physical examination; or oculomotor function on vestibulo-oculomotor examination.

Sleep disorder: A disorder of sleep that includes, but is not limited to, insomnia, disorders of daytime somnolence, parasomnias, chronobiological disorders, and sleep consequences of neurological disorders. Non-limiting examples of sleep disorders include rapid eye movement behavior disorder, restless legs syndrome, periodic leg movements of sleep, obstructive sleep apnea, central sleep apnea, nightmares, sleep tenors, sleepwalking, confusional arousals, sleep paralysis, sleep eating disorder, or narcolepsy (See, for example, C G Goetz (editor), Textbook of Clinical Neurology, 3^{rd} Edition, 2007, Chapter 54)

Sleep disturbance: An observed or reported alteration in the initiation, maintenance, or quality of sleep that may be a symptom or sign of a medical, neurological, or psychiatric disorder. A sleep disturbance also may be a symptom or sign of a sleep disorder.

Sleep Terrors: An awakening from sleep characterized by intense anxiety upon awakening. Sleep terrors can be differentiated from nightmares because there is significantly less recall of frightening dream content in sleep tenors. Sleep terrors may be present as a sign or symptom of another psychiatric disorder. Sleep terrors can be difficult to distinguish from nocturnal panic attacks.

Stimulant or Stimulatory agent: A class of medication intended to increase arousal, wakefulness, attention, concentration, or cognition. Non-limiting examples of stimulants include caffeine, methylxanthine, paraxanthine, theobromine, theophylline, mazindol, pipradrol, sibutramine, pemoline, methylphenidate, amphetamine, methamphetamine, mixed amphetamine salts, modafinil, or any prodrug or pharmacologically active metabolite thereof. In an example, a stimulatory agent is a central nervous system stimulatory agent, such as an amphetamine, methylphenidate, pemoline, caffeine, a centrally acting α-1 agonist, dextroamphetamine or modafinil. In some examples, the disclosed methods include administering a therapeutically effective concentration of tizanidine in the absence of a stimulatory agent.

Subject: Multi-cellular organisms, to include human and non-human primates, non-primate vertebrates, and non-vertebrate animals.

Symptom: A problem, complaint, or issue reported by a subject that is primarily a subjective complaint. Pain, fatigue, or changes in mood are commonly reported symptoms. Symptoms are distinguished from signs in that signs typically can be confirmed with objective evidence such as observation, tests or examinations, whereas symptoms rely upon the subject's self-report.

Therapeutically effective amount: An amount of a pharmaceutical preparation such as tizanidine that alone, or together with a pharmaceutically acceptable carrier or one or more additional therapeutic agents, induces the desired response. A therapeutic agent, such as tizanidine, is administered in therapeutically effective amounts.

Effective amounts of a therapeutic agent can be determined in many different ways, such as by monitoring a sign or symptom of a psychiatric disorder (e.g., a sleep disorder, mood disorder, perceptual disturbance, and/or anxiety disorder).

Therapeutic agents can be administered in a single dose, or in several doses, for example daily or twice daily, during a course of treatment. However, the effective amount of can be dependent on the source applied, the subject being treated, the severity and type of the condition being treated, and the manner of administration.

In one example, it is an amount sufficient to reduce or inhibit a sign or symptom of a disorder, such as a sign or symptom of a psychiatric disorder (e.g., a sleep disorder, mood disorder, perceptual disturbance, or anxiety disorder). For example, a pharmaceutical preparation can decrease a sign or symptom of a psychiatric disorder by at least 20%, at least 50%, at least 70%, at least 90%, at least 98%, or even at least 100%, as compared to the sign or symptom observed in the absence of the pharmaceutical preparation.

Tizanidine: A centrally acting α2 adrenergic agonist and/or imidazoline agonist that is commonly used to treat the spasms, cramping, and tightness of muscles caused by medical problems such as multiple sclerosis, spastic diplegia, back pain, or certain other injuries to the spine or central nervous system. Tizanidine is a short-acting medication with peak efficacy within two to four hours after an oral dose depending upon the specific oral formulation. Tizanidine serum levels may vary by the specific oral formulation administered (capsule or tablet) and if the capsule or tablet is taken with food. Generally, peak serum levels are about 2.7 ng/ml after a single 4 mg tablet and 4.0 ng/ml after a single 4 mg capsule. As used herein, tizanidine is administered to reduce or inhibit a symptom associated with a psychiatric disorder and/or a neurologic disorder where the symptoms include, but are not limited to, a sleep disorder, anxiety disorder, perceptual disturbance or mood disorder. Tizanidine is commercially available (e.g., ZANAFLEX® or SIRDALUD®).

Traumatic Brain Injury (TBI): A disorder caused by an injury to the head which results in a post-injury disturbance in mood, anxiety, cognitive function, pain, balance, oculomotor function, level of consciousness, or memory. As used in this document, TBI is inclusive of all reported injury mechanisms (penetrating, blast, blunt), any post-injury changes, the inflammatory—immunologic response commonly observed after injury or illness, or any iatrogenic causes or consequences. Examples of iatrogenic causes or consequences include changes in the brain as a result of surgery, chemotherapy for cancer, radiation therapy, or a medication side-effect.

Treating: “Treating” or “Treatment” refers to a pharmaceutical product that partially or completely ameliorates a sign or symptom of a disorder, such as a sign or symptom of a psychiatric disorder (e.g., a sleep disorder, mood disorder, perceptual disturbance, or anxiety disorder) or a sign or symptom of a neurological or medical disorder (e.g., depressed or sad mood due to a neurological illness; anxiety occurring after a traumatic brain injury).

### II. Overview of Several Embodiments

Disclosed herein are methods of treating a psychiatric disorder, such as a sleep disorder, an anxiety disorder, a mood disorder or a perceptual disturbance. In one example, the method includes selecting a subject with a sleep disturbance or anxiety disorder in the absence of an underlying physical disorder and administering a therapeutically effective amount of tizanidine, thereby reducing or inhibiting a symptom of the sleep disorder or anxiety disorder. In one example, a sleep disturbance includes nightmares. In other examples, the sleep disturbance or anxiety disorder is associated with a neurological or psychiatric disorder. For example, the neurological or psychiatric disorder includes PTSD, Parkinson's disease, a brain injury or a partial complex seizure disorder.

In certain examples, administering a therapeutically effective amount of tizanidine to treat a sleep disorder or anxiety disorder includes administering about 2 mg to about 20 mg of tizanidine, such as about 4 mg of tizanidine. In an example, tizanidine is formulated for oral administration, such as a time-release formulation which releases about 5% of the original dose per hour. In one example, tizanidine is formulated as a time-release formulation which releases about 8% of the original dose per hour. In some embodiments, administering a therapeutically effective amount of tizanidine includes administering tizanidine daily. In other embodiments, administering a therapeutically effective amount of tizanidine includes administering tizanidine twice daily.

In one embodiment, a method of treating a sleep disorder or anxiety disorder further includes administering a therapeutically effective amount of an antidepressant, stimulant, antipsychotic, mood stabilizer, anticonvulsant, benzodiazepine, or a combination thereof. For example, the antidepressant the antidepressant comprises one or more serotonergic antidepressant medication, mirtazapine, trazodone, atomoxetine, bupropion, a tricyclic antidepressant or a combination thereof.

In another embodiment, a method of treating a sleep disorder or anxiety disorder includes administering a therapeutically effective amount of tizanidine in the absence of or without a stimulatory agent, such as a central nervous system stimulatory agent.

In one embodiment, a method of treating a mood disorder or a perceptual disturbance is disclosed. For example, the method includes selecting a subject with a mood disorder or perceptual disturbance. The method also includes administering a therapeutically effective amount of tizanidine, thereby reducing or inhibiting a symptom of the mood disorder or perceptual disturbance. In one example, the mood disorder includes a depressive disorder, an elevated mood, a manic mood, an irritable mood or a combination thereof. In an example, the perceptual disturbance includes a visual disturbance, an auditory disturbance, an olfactory disturbance, a dissociative state or a combination thereof. In an example, the method of treating a mood disorder or a perceptual disturbance includes administering a therapeutically effective amount of tizanidine in the absence of or without a stimulatory agent, such as a central nervous system stimulatory agent.

In certain examples, administering a therapeutically effective amount of tizanidine to treat a mood disorder or perceptual disorder includes administering about 2 mg to about 20 mg of tizanidine, such as about 4 mg of tizanidine. In an example, tizanidine is formulated for oral administration, such as a time-release formulation which releases about 5% of the original dose per hour. In one example, tizanidine is formulated as a time-release formulation which releases about 8% of the original dose per hour. In some embodiments, administering a therapeutically effective amount of tizanidine includes administering tizanidine daily. In other embodiments, administering a therapeutically effective amount of tizanidine includes administering tizanidine twice daily.

In one embodiment, a method of treating a mood disorder or perceptual disorder further includes administering a therapeutically effective amount of an antidepressant, a benzodiazepine, a non-benzodiazepine hypnotic, stimulant medication, typical or atypical antipsychotic medication, NMDA antagonist, mood stabilize, anticonvulsant, buspirone, droperidol, or a combination thereof. For example, the antidepressant includes one or more of a serotonergic antidepressant medication, mirtazapine, trazodone, atomoxetine, bupropion, a tricyclic antidepressant or a combination thereof.

### III. Methods of Treatment

Alpha-2-adrenergic agonists (such as clonidine) can be used to treat the following conditions: hypertension; alcohol withdrawal; opiate withdrawal; sedation prior to surgical procedures; attention-deficit hyperactivity disorder; anxiety and/or behavioral problems due to other psychiatric disorders. For example, clonidine and guanfacine, two α2-adrenergic agonists are used to treat certain psychiatric disorders. These compounds were originally introduced as treatments for high blood pressure. Clonidine and guanfacine are known to those skilled in the relevant art as commonly prescribed medications for treating high blood pressure. Other α2 adrenergic agonists, such as tizanidine, are commonly used to treat the spasms, cramping, and tightness of muscles caused by medical problems such as multiple sclerosis, spastic diplegia, back pain, or certain other injuries to the spine or central nervous system. Although tizanidine and clonidine have similar chemical structures, tizanidine has been shown to have only one-tenth to one-fiftieth the potency of clonidine in lowering blood pressure. Tizanidine may affect spastic muscles through the imidazoline receptor in addition to the alpha-2 adrenergic receptor. Tizanidine is known to those skilled in the relevant art as a medication that can reduce muscle spasticity; in particular, tizanidine is useful for reducing the muscle spasticity due to what are commonly called upper motor neuron lesions. Example disorders than can produce upper motor neuron lesions include multiple sclerosis, acquired brain injury, spinal cord injury, or stroke. Tizanidine is also known to those skilled in the relevant art as a useful medication for pain, including myofascial pain, back pain, and trigeminal neuralgia. Tizanidine is not known to be useful for treating high blood pressure. Although α2-adrenergic agonists (such as clonidine) can be used to reduce signs and symptoms of psychiatric disorders, this use is often limited due to their undesirable side-effects on blood pressure and heart rate. Frequently, the α2-adrenergic agonist either cannot be tolerated by the subject, or, it must be used in smaller than desired doses; either outcome deprives the patient of a potentially useful therapeutic benefit. The α2-adrenergic agonist effect in lowering blood pressure is a particular problem in subjects without a pathologic condition that elevates blood pressure (e.g., essential hypertension). Therefore, a need exists to identify therapeutic agents capable of reducing signs and symptoms of psychiatric disorder without causing such undesirable side effects.

Another α2-adrenergic agonist medication, guanfacine, was shown to be ineffective in relieving signs or symptoms of PTSD in a placebo-controlled clinical trial. In this trial guanfacine was administered at an average dose of 2.4 mg to military veterans with PTSD in two groups: veterans taking antidepressant medication and veterans not taking antidepressant medication. Guanfacine was not effective compared to placebo.

It is shown herein for the first time that tizanidine can be used to treat a psychiatric disorder such as PTSD, anxiety, depression, psychosis or impaired cognition after commonly-utilized treatments had been demonstrated ineffective. Based on these observations, new methods of treating psychiatric disorders, such as a sleep disturbance, anxiety, mood disorder or a perceptual disturbance are disclosed.

The subject can be any subject of interest. The disclosed methods of treating a psychiatric disorder can include selecting a subject with a psychiatric disorder, such as a sleep disorder, mood disorder, perceptual disturbance, or anxiety disorder in the absence of an underlying physical disorder. In other embodiments, the method includes selecting a subject that does not have a traumatic brain injury. In yet other embodiments, the method includes selecting a subject that does not have a physical sign or symptom involving muscle spasticity or rigidity. However, in other embodiments, the method includes selecting a subject that has a TBI.

In even further embodiments, the method includes selecting a subject that has PTSD. In one particular example, the method includes selecting a subject that has PTSD and does not have a physical sign or symptom involving muscle spasticity or rigidity. In some examples, the method includes selecting a subject that has PTSD, but does not have a sleeping disorder. In one example, the method includes selecting a subject that has PTSD and does not have an underlying physical disorder.

In one example, the method includes selecting a subject with a psychiatric disorder. For example, in one embodiment the method includes selecting a subject with a sleep disturbance or anxiety disorder in the absence of an underlying physical disorder and administering a therapeutically effective amount of tizanidine, thereby reducing or inhibiting a symptom of the sleep disorder or anxiety disorder. In other embodiments, the method includes selecting a subject with a mood disorder or perceptual disturbance. For example, the method includes selecting a subject with a mood disorder or perceptual disturbance in the absence of a physical disorder. In one example, selecting a subject with a sleep disturbance or anxiety disorder in the absence of an underlying physical disorder includes selecting a subject without a traumatic brain injury.

In some examples, subjects are initially screened to determine if they have one or more symptoms associated with a sleep disorder, anxiety disorder, mood or perceptual disorder. For example, the diagnostic methods known to those of ordinary skill in the art, including psychological and neurological evaluations, can be used to screen subjects to determine if they are candidates for the disclosed therapies.

The disclosed methods include administering a therapeutically effective amount of tizanidine to treat the psychiatric disorder to reduce or inhibit a symptom of the disorder. The psychiatric disorder can be a sleep disorder, anxiety disorder, a mood disorder or perceptual disorder. Reduction of a symptom of the disorder can include decrease in a sign or symptom of a psychiatric disorder by at least 20%, at least 50%, at least 70%, at least 90%, at least 98%, or even at least 100%, as compared to the sign or symptom observed in the absence of tizanidine administration.

The methods can include administering tizanidine in combination with other therapeutic agents. For example, additional therapeutic agents such as an antidepressant, antipsychotic, mood stabilizer, anticonvulsant, benzodiazepine, a non-benzodiazepine-hypnotic, a stimulant medication, a typical or atypical antipsychotic medication, an NMDA antagonist, buspirone, droperidol, in combination with tizanidine.

Methods of administration of the disclosed agents are routine, and can be determined by a skilled clinician. For example, tizanidine can be administered orally, via injection, topically, transdermally, parenterally, or via inhalation or spray. In a particular example, an agent including tizanidine is administered orally to a mammalian subject, such as a human.

A therapeutic effective concentration of tizanidine is a concentration that when administered induces the desired response (e.g., reduce or inhibit one or more symptoms associated with a psychiatric disorder). Effective amounts of a tizanidine can be determined in many different ways, such as by monitoring a sign or symptom of a psychiatric disorder (e.g., a sleep disorder, mood disorder, perceptual disturbance, or anxiety disorder). Tizanidine can be administered in a single dose, or in several doses, for example daily or twice daily, during a course of treatment. However, the effective amount of tizanidine can be dependent on the source applied, the subject being treated, the severity and type of the condition being treated, and the manner of administration. Commercially available sources of tizanidine, such as ZANAFLEX® or SIRDALUD® can be used.

In one example, it is an amount sufficient to reduce or inhibit a sign or symptom of a disorder, such as a sign or symptom of psychiatric disorder (e.g., a sleep disorder, mood disorder, perceptual disturbance, or anxiety disorder). For example, a pharmaceutical preparation can decrease a sign or symptom of a psychiatric disorder by at least 20%, at least 50%, at least 70%, at least 90%, at least 98%, or even at least 100%, such as about 25% to about 90%, about 30% to about 80%, to about 40% to about 60%, including about 25%, 30%, 35%, 40%, 50%, 55%, 60%, 65%, 70%, 75%, 80%, 85%, 90%, 95% or 98% as compared to the sign or symptom observed in the absence of tizanidine.

The therapeutically effective amount of the agents administered can vary depending upon the desired effects and the subject to be treated. In one example, the method includes daily administration of at least 2 mg of tizanidine to the subject (such as a human subject). For example, a human can be administered at least 2 mg to at least 50 mg of the agent daily, such as about 2 mg to about 30 mg daily, about 4 mg to about 20 mg daily, such as about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg. In an example, the subject is administered at least 2 mg (such as 4-20 mg) orally of tizanidine. In some embodiments, administering a therapeutically effective amount of tizanidine includes administering tizanidine twice daily, such as at least 2 mg twice daily, such as at least about 4 mg, about 5 mg, about 6 mg, about 7 mg, about 8 mg, 9 about mg, about 10 mg, about 11 mg, about 12 mg, about 13 mg, about 14 mg, about 15 mg, about 16 mg, about 17 mg, about 18 mg, about 19 mg, or about 20 mg twice daily.

In an example, tizanidine is formulated for oral administration, such as a time-release formulation which releases at least about 3%, such as about 4%, about 5%, about 6%, about 7%, about 8%, about 9%, about 10%, or about 12%, of the original dose per hour. In certain examples, tizanidine is formulated as a time-release formulation which releases about 5% of the original dose per hour, such as about 5% of the original tizanidine dose each hour in a 20 to 24-hour period. In other examples, tizanidine is formulated as a time-release formulation which releases about 8%, of the original dose per hour, such as about 8%, of the original tizanidine dose each hour in a 12-hour period In further examples, a single dose containing about 8, about 12 or about 16 mg of tizanidine is formulated as a time-release to be released over 24 hour period. In another certain example, tizanidine is formulated as a time-release formulation which releases about 8%, of the original dose per hour.

In one example, the method includes daily administration of at least 1 μg of tizanidine to the subject (such as a human subject). For example, a human can be administered at least 1 μg or at least 1000 mg of the agent daily, such as 10 μg to 100 μg daily, 100 μg to 1 mg daily, 100 μg 1000 mg for example 100 μg daily, 1 mg daily, 10 mg daily, 100 mg daily, or 1000 mg daily.

In an example, the subject is administered at least 1 μg (such as 1-100 μg) intravenously of tizanidine. In one example, the subject is administered at least 1 mg intramuscularly (for example in an extremity) of such composition. The dosage can be administered in divided doses (such as 2, 3, or 4 divided doses per day), or in a single dosage daily. In a specific example, the subject is administered at least 0.15 mg per kg of body weight of the agent approximately every four weeks for at least 6 months. For example, 0.15 mg/kg, 0.5 mg/kg, 2 mg/kg, 3 mg/kg, 5 mg/kg or 6 mg/kg is administered, such as via intravenous, intramuscular, or subcutaneous injections, or an intrathecal infusion, every 28 days for 6 months. In an example, a human is administered tizanidine to maintain a serum concentration of about 1 ng/ml to about 24 ng/ml, such as about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 15 ng/ml, 20 ng/ml or about 23 ng/ml. For example, a subject is administered tizanidine at a rate of about 3 mg/24 hours, 4 mg/24 hours, 5 mg/24 hours, 16 mg/24 hours, 24 mg/24 hours, 32 mg/24 hours, or 48 mg/24 hours in order to maintain a serum concentration of about 1 ng/ml to about 24 ng/ml.

In other examples, the subject is administered at least 4 mg per 24 hours (4 mg/24 hours) of tizanidine transdermally, such as via a transdermal patch. In a specific example, a transdermal patch would deliver tizanidine at a rate of about 3 mg/24 hours, 4 mg/24 hours, 5 mg/24 hours, 16 mg/24 hours, 24 mg/24 hours, 32 mg/24 hours, or 48 mg/24 hours. In another specific example, a transdermal delivery system would be intended to maintain a serum concentration of about 4 nanograms/milliliter (ng/ml), about 1 ng/ml, about 2 ng/ml, about 3 ng/ml, about 5 ng/ml, about 6 ng/ml, about 7 ng/ml, about 8 ng/ml, about 9 ng/ml, about 10 ng/ml, or about 15 ng/ml.

In particular examples, the subject is administered tizanidine on a multiple daily dosing schedule, such as at least two consecutive days, 10 consecutive days, and so forth, for example for a period of weeks, months, or years. In one example, the subject is administered the agent daily for a period of at least 30 days, such as at least 2 months, at least 4 months, at least 6 months, at least 12 months, at least 24 months, or at least 36 months.

This disclosure includes within its scope pharmaceutical compositions including tizanidine formulated for use in human or veterinary medicine. While tizanidine formulations typically will be used to treat human subjects, they also can be used to treat similar or identical diseases in other vertebrates, such as other primates, dogs, cats, horses, and cows.

Pharmaceutical compositions that include tizanidine as described herein as an active ingredient, or that include both tizanidine and an additional agent as active ingredients, can be formulated with an appropriate solid or liquid carrier, depending upon the particular mode of administration chosen. A suitable administration format can best be determined by a medical practitioner for each subject individually. Various pharmaceutically acceptable carriers and their formulation are described in standard formulation treatises, for instance, Remington's Pharmaceutical Sciences by E. W. Martin Mack Publishing Co., Easton, Pa., 19th Edition (1995). See also Wang & Hanson (1988) Journal of Parenteral Science and Technology, Technical Report No. 10, Supp. 42: 2S. For example, a suitable pharmacological composition can be formulated to facilitate the use of tizanidine in vivo. Such a composition can be suitable for delivery of the active ingredient to any suitable host, such as a patient for medical application, and can be manufactured in a manner that is itself known, for instance, by means of conventional mixing dissolving, granulating, dragee-making, levigating, emulsifying, encapsulating, entrapping, or lyophilizing processes.

The dosage form of the pharmaceutical composition is determined by the mode of administration chosen. For instance, in addition to oral formulations, injectable fluids, inhalational, and transdermal formulations can be employed. Oral formulations can be liquid (for instance, syrups, solutions, or suspensions), or solid (for instance, powders, pills, tablets, or capsules). For solid compositions, conventional non-toxic solid carriers can include pharmaceutical grades of mannitol, lactose, starch, or magnesium stearate. Inhalational preparations can include aerosols, particulates, and the like. In general, the goal for particle size for inhalation is about 1 μm or less in order that the pharmaceutical reach the alveolar region of the lung for absorption. Actual methods of preparing such dosage forms are known, or will be apparent, to those of ordinary skill in the art.

The compositions or pharmaceutical compositions can be administered by any route, including parenteral administration, for example, intravenous, intraperitoneal, intramuscular, intraperitoneal, intrathecal, or intra-articular injection or infusion, or by sublingual, oral, topical, intra-nasal, or transmucosal administration, or by pulmonary inhalation. When tizanidine compositions are provided as parenteral compositions, for instance, for injection or infusion, they are generally suspended in an aqueous carrier, for example, in an isotonic buffer solution at a pH of about 3.0 to about 8.0, for example at a pH of about 3.5 to about 7.4, 3.5 to 6.0, or 3.5 to about 5.0. Useful buffers include sodium citrate-citric acid and sodium phosphate-phosphoric acid, and sodium acetate/acetic acid buffers.

For oral administration, the pharmaceutical compositions that include tizanidine can take the form of, for example, tablets or capsules prepared by conventional means with pharmaceutically acceptable excipients such as binding agents (for instance, pregelatinised maize starch, polyvinyl pyrrolidone or hydroxypropyl methylcellulose); fillers (for instance, lactose, microcrystalline cellulose or calcium hydrogen phosphate); lubricants (for instance, magnesium stearate, talc or silica); disintegrants (for instance, potato starch or sodium starch glycolate); or wetting agents (for instance, sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (for instance, sorbitol syrup, cellulose derivatives or hydrogenated edible fats); emulsifying agents (for instance, lecithin or acacia); non-aqueous vehicles (for instance, almond oil, oily esters, ethyl alcohol or fractionated vegetable oils); and preservatives (for instance, methyl or propyl-p-hydroxybenzoates or sorbic acid). The preparations can also contain buffer salts, flavoring, coloring, and sweetening agents as appropriate.

For administration by inhalation, tizanidine compositions for use according to the present disclosure are conveniently delivered in the form of an aerosol spray presentation from pressurized packs or a nebulizer, with the use of a suitable propellant, for instance, dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges for use in an inhaler or insufflator can be formulated containing a powder mix of the compound and a suitable powder base such as lactose or starch.

In specific examples, the agent for administration can include a solution of tizanidine dissolved in a pharmaceutically acceptable carrier, such as an aqueous carrier. A variety of aqueous carriers can be used, for example, buffered saline and the like. These solutions are sterile and generally free of undesirable matter. These agents may be sterilized by conventional, well known sterilization techniques. The agents may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of tizanidine in these formulations can vary widely, and will be selected primarily based on fluid volumes, viscosities, body weight and the like in accordance with the particular mode of administration selected and the subject's needs. Actual methods for preparing administrable agents will be known or apparent to those skilled in the art and are described in more detail in such publications as Remington's Pharmaceutical Science, 19th ed., Mack Publishing Company, Easton, Pa. (1995).

The disclosed agents including tizanidine may be provided in lyophilized form and rehydrated with sterile water before administration, although they are also provided in sterile solutions of known concentration. The agent solution is then added to an infusion bag containing 0.9% Sodium Chloride, USP, and typically administered at a dosage of from 0.5 to 15 mg/kg of body weight. Considerable experience is available in the art in the administration of compounds such as tizanidine. These drugs can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

The disclosed agents and compositions including tizanidine can further include one or more biologically active or inactive compounds (or both), such as antidepressants and conventional non-toxic pharmaceutically acceptable carriers, respectively. Examples of such biologically inactive compounds include, but are not limited to: carriers, thickeners, diluents, buffers, preservatives, and carriers. The pharmaceutically acceptable carriers useful for these formulations are conventional (see Remington's Pharmaceutical Sciences, by E. W. Martin, Mack Publishing Co., Easton, Pa., 19th Edition (1995)). In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can include minor amounts of non-toxic auxiliary substances, such as wetting or emulsifying agents, preservatives, and pH buffering agents and the like, for example sodium acetate or sorbitan monolaurate.

Controlled release parenteral formulations can be made as implants, oily injections, or as particulate systems. For a broad overview of protein delivery systems see, Banga, Therapeutic Peptides and Proteins: Formulation, Processing, and Delivery Systems, Technomic Publishing Company, Inc., Lancaster, Pa., (1995) incorporated herein by reference. Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, Colloidal Drug Delivery Systems, ed., Marcel Dekker, Inc., New York, N.Y., pp. 219-342 (1994); and Tice & Tabibi, Treatise on Controlled Drug Delivery, ed., Marcel Dekker, Inc. New York, N.Y., pp. 315-339, (1992) both of which are incorporated herein by reference.

Polymers can be used for ion-controlled release of the agents disclosed herein. Various degradable and nondegradable polymeric matrices for use in controlled drug delivery are known in the art (Langer, Accounts Chem. Res. 26: 537-542, 1993). For example, the block copolymer, polaxamer 407, exists as a viscous yet mobile liquid at low temperatures but forms a semisolid gel at body temperature. Alternatively, hydroxyapatite has been used as a microcarrier for controlled release of proteins (Ijntema et al., Int. J. Pharm., 112: 215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known (see U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342 and U.S. Pat. No. 5,534,496).

Selecting a Subject In some examples, the disclosed method of treating a psychiatric disorder includes selecting a subject with a psychiatric disorder, such as a sleep disorder, mood disorder, perceptual disturbance, or anxiety disorder in the absence of an underlying physical disorder. An underlying physical disorder includes a disorder that is reduced or prevented by treatment with a centrally acting α2 adrenergic or imidazoline agonist, such as tizanidine. For example, the method includes selecting a subject that does have a disorder or condition that includes spasms, cramping, and tightness of muscles caused by medical problems such as multiple sclerosis, spastic diplegia, back pain, or other certain injuries to the spine or central nervous system that could be relieved or prevented by treatment with a centrally acting α2 adrenergic agonist (e.g., tizanidine).

In other examples, the method includes selecting a subject who has not had a TBI. Thus the subject has not had traumatic brain injury as a result of any injury mechanisms (penetrating, blast, blunt), any post-injury changes, the inflammatory—immunologic response commonly observed after injury or illness, or any iatrogenic causes or consequences.

In yet other examples, a subject is selected that does not have a physical sign or symptom involving muscle spasticity or rigidity.

### Screening Subjects

Subjects can be screened prior to initiating the disclosed therapies, for example to select a subject in need of treatment. In an example, a subject in need of the disclosed therapies is selected according to the criteria set forth in the American Psychiatric Association Diagnostic and Statistical Manual, Fourth Edition, (DSM-IV) based upon symptoms reported by the subject. In some examples, psychological and/or neuropsychological testing can be used to identify or confirm previously identified psychiatric disorders. Non-limiting examples of psychological testing include the following tests: BDI-II; DTS (a measure of PTSD); and MMPI-2. Exemplary neuropsychological tests include the following: WTAR; Digit Symbol-Coding, Digit Span, and Block Design subtests from WAIS-III; CVLT-II; BVMT-R; RCFT; Ruff 2 & 7 Selective Attention Test; Trialmaking (parts A & B); phonemic fluency (letters CFL);

category fluency (animals); WCST; Stroop Color and Word Test; Grooved Pegboard; Rey 15-Item Memory Test; and other methods known methods known to those of ordinary skill in the art.

The detection of a psychiatric disorder, such as a sleep disorder, mood disorder, anxiety disorder and/or perceptual disorder, by the DSM-IV criteria and/or psychological and neurological tests indicates that the disorder can be treated using the methods provided herein.

### Additional Pharmaceutical Agents

In particular examples, prior to, during, or following administration of tizanidine, the subject can receive one or more other additional pharmaceutical agents. In one example, the subject receives one or more agents to reduce or inhibit one or more symptoms associated with one or more psychiatric disorders.

Antidepressant agents can be administered in combination with tizanidine to treat one or more symptoms associated with depression. In an embodiment, an antidepressant agent is an agent with established efficacy in treating a mood disorder, as exhibited by FDA marketing approval for mood disorder treatment and/or clinical trial data showing a reduction in target signs or symptoms of depression, regardless of the underlying cause of such depressive signs or symptoms. An antidepressant agent can also be an agent that is a member of one of the following drug classes: serotonin-specific re-uptake inhibitor (SSRI); serotonin-norepinephrine re-uptake inhibitor (SNRI); serotonin-dopamine re-uptake inhibitor; tricyclic antidepressant; or monoamine oxidase inhibitor. For example, an antidepressant can be one or more of a serotonergic antidepressant medication such as SSRI, SNRI, nefazodone, or mirtazapine; trazodone; atomoxetine; bupropion; or tricyclic antidepressant, a benzodiazepine, a non-benzodiazepine hypnotic, a stimulant medication, typical or atypical antipsychotic medication, an NMDA antagonist, a mood stabilizer, anticonvulsant, buspirone, droperidol, or a combination thereof. Non-limiting examples of these medications include: SSRI (sertraline, fluoxetine, paroxetine, citalopram, escitalopram, fluvoxamine, zimelidine, or dapoxetine); SNRI (venlafaxine, desvenlafaxine, duloxetine, milnacipran, or bicifadine); tricyclic antidepressant (nortriptyline, amitriptyline, desipramine, imipramine, protriptyline, or clomipramine); MAO inhibitor (phenelzine, moclobemide, or selegiline); stimulant (caffeine, methylxanthine, paraxanthine, theobromine, theophylline, mazindol, pipradrol, sibutramine, pemoline, methylphenidate, amphetamine, methamphetamine, mixed amphetamine salts, or modafinil); NMDA antagonist (ketamine, amantadine, memantine, dextromethorphan, or riluzole); benzodiazepine (clonazepam, alprazolam, diazepam, clorazepate, lorazepam, oxazepam, midazolam, estazolam, triazolam, flurazepam, nitrazepam, prazepam, or chlordiazepoxide); non-benzodiazepine hypnotic (zolpidem, zaleplon, zopiclone, eszopiclone, or ramelteon); typical antipsychotic medication (haloperidol, chlorpromazine, mesoridazdine, periciazine, perphenazine, trifluoperazine, thiothixene, zuclopenthixol pimozide, loxapine, fluphenazine, thioridazine, or molindone); atypical antipsychotic medication (aripiprazole, olanzapine, risperidone, paliperidone, quetiapine, ziprasidone, melperone, sertindole, iloperidone, asenapine, amisulpride, zotepine, or clozapine); mood stabilizer and/or anticonvulsant (lithium in various salt formulations; valproic acid in various formulations; carbamazepine; phenytoin and fosphenytoin; lamotrigine; tiagibine; pregabalin; or gabapentin).

Mood stabilizer agents can be administered in combination with tizanidine to treat one or more symptoms associated with a mood disorder. A mood stabilizer agent can be any agent with established efficacy in treating a mood disorder, as exhibited by FDA marketing approval for mood disorder treatment and/or clinical trial data showing a reduction in target signs or symptoms of mania, hypomania, irritable mania, or depression, regardless of the underlying cause of the signs or symptoms. A mood stabilizing agent can also be an agent that is a member of one of the following drug classes: lithium-containing compounds or anti-convulsants (e.g., any medication approved by FDA as single or adjunct therapy for any type of seizures or epilepsy disorder). In one embodiment, a mood stabilizing agent is an agent including one or more of the following compounds: LiCO₃, Lithium Citrate, valproic acid, divalproex, other valproic acid or divalproex derivatives, lamotrigine, carbamazepine, oxcarbazepine, phenytoin, fosphenytoin, topiramate, pregabalin, or tiagibine.

Antipsychotic agents can be administered in combination with tizanidine to treat one or more symptoms associated with a psychotic disorder. An antipsychotic agent can be any agent with established efficacy in treating a psychotic disorder, as exhibited by FDA marketing approval for psychosis or any psychotic disorder, and/or clinical trial data showing a reduction in target signs or symptoms of psychosis regardless of the underlying cause of the signs or symptoms. In one embodiment, an antipsychotic agent is a butyrophenone compound. In some embodiments, an antipsychotic agent is an agent including any neuroleptic, phenothiazine or other type of medication that partially or fully blocks the action of dopamine at a dopamine receptor site.

Anxiolytic or hypnotic agents can be administered in combination with tizanidine to treat one or more symptoms associated with an anxiety or sleep disorder. An anxiolytic or hypnotic agent can be any agent with established efficacy in treating an anxiety or sleep disorder, as exhibited by FDA marketing approval for any anxiety or sleep disorder, and/or clinical trial data showing a reduction in target signs or symptoms of any anxiety or sleep disorder, regardless of the underlying cause of the signs or symptoms. In one embodiment, an anxiolytic or hypnotic agent is a member of one of the following drug classes: benzodiazepine, barbiturate, melatonin receptor agonist, ethanol, serotonin receptor 5HT1A agonist, serotonin receptor 5HT2 or 5HT3 antagonist, α2 adrenergic agonist, or α1 antagonist. In some embodiments, an anxiolytic or hypnotic agent is any agent that partially or fully mimics or enhances the action of gamma-aminobutyric acid (GABA) at a GABA receptor site. In another embodiment, an anxiolytic agent is any agent that partially or fully mimics, enhances, diminishes, or blocks the action within the central nervous system of any compound with the characteristic four fused rings of a steroid.

Acetylcholinesterase inhibitors can be administered in combination with tizanidine to inhibit, either reversibly or irreversibly, the action of acetylcholinesterase or any enzyme that metabolizes acetylcholine.

Dopamine agonists can be administered in combination with tizanidine to activate, either partially or completely, a dopamine receptor. Non-limiting examples of dopamine agonists include amantadine, L-Dopa; compositions containing L-Dopa, bromocriptine, cabergoline, pramipexole, ropinirole, apomorphine, or rotigotine.

Specific, non-limiting examples of adjunctive agents by psychiatric disorder category include: (1) Mood Disorders—thyroid hormone (thyroxine or triiodothyronine), lithium, caffeine, buspirone, stimulants (non-limiting examples of stimulants include: methylphenidate, dextroamphetamine, amphetamine, mixed amphetamine salt compounds, modafinil; plus any prodrugs or enantiomers of these stimulant compounds), and NMDA antagonists (non-limiting examples of NMDA antagonists include: ketamine, dextromethorphan/dextrorphan, amantadine, riluzole, memantine); (2) Cognitive Disorders—NMDA antagonists (non-limiting examples of NMDA antagonists include: ketamine, dextromethorphan/dextrorphan, amantadine, riluzole, memantine), stimulants; and (3) Anxiety Disorders—α1 antagonist, α2 agonist, anticonvulsants, atypical antipsychotics, and stimulants.

The following examples are provided to illustrate certain particular features and/or embodiments. These examples should not be construed to limit the disclosure to the particular features or embodiments described.

### EXAMPLES

### Example 1

This example provides six case summaries that illustrate the effective use of tizanidine as an adjunctive treatment for PTSD, anxiety, depression, psychosis or impaired cognition after commonly-utilized treatments had been demonstrated ineffective.

### Case Summary 1:

The subject was a twenty-five year old Army soldier admitted to the psychiatric ward of a hospital when home on leave from active duty in Iraq. He was a truck driver. Approximately two months prior to hospital admission, the soldier was ejected from the cab of a military heavy truck he was driving by a large explosion. The explosion killed the assistant driver of the vehicle and tossed the driver across several lanes of traffic. While home on mid-tour leave six weeks later, he was admitted for treatment of severe anxiety.

At the time of hospital admission, anxiety caused him to be unable to leave the home or drive a car. Prominent signs and symptoms of PTSD included: severe anxiety that has worsened since leaving Iraq; initial and middle insomnia; daily nightmares of the explosion that injured him and killed the assistant driver; multiple flashbacks of the accident, which included auditory hallucinations of the deceased assistant driver screaming; avoidance of military weapons; extreme irritability; easily startled; psychological and physiological reaction to reminders of combat (e.g., anxiety when seeing trash on the road (a common means of disguising roadside bombs in Iraq) or people who appear to be of Middle-Eastern descent). He also reported increased alcohol use when home on leave. The soldier endorsed signs and symptoms of major depression as depressed/irritable mood; poor appetite; decreased energy; poor concentration; and psychomotor agitation.

Treatment was begun with sertraline 100 mg per day for anxiety and nortriptyline 25 mg at bedtime for headaches. After four weeks, the subject reported only a modest decrease in anxiety, depression, and psychosis. The intensity and frequency of nightmares were unchanged. Treatment with tizanidine was started and the dose was increased to 8 mg at bedtime. After two weeks of taking tizanidine, the subject reported an approximate 50% reduction in nightmares, and less daytime anxiety.

### Case Summary 2:

The subject was a twenty-five year old military veteran who completed two tours of duty in Iraq and suffered from PTSD and Panic Disorder. He was first evaluated approximately one year after his last tour in Iraq. At that time, the veteran reported a prior diagnosis of PTSD while in Iraq. The evaluating psychiatrist noted significant signs and symptoms of anxiety, including both PTSD and Panic Disorder. The veteran reported being involved in numerous firefights with enemy forces during his two tours of duty. One particular trauma was discovering a fatally wounded fellow Marine who had been severely wounded by a rocket-propelled grenade.

Treatment of the subject with sertraline was started. Approximately six months later, the veteran was seen for a follow-up visit. At that time, he reported worsening anxiety, more frequent panic attacks, and near-daily nightmares. He also disclosed experiencing near-daily auditory and visual hallucinations of war-related events. Sertraline was increased to 200mg daily and prazosin 5 mg at bedtime was added for nightmares. These medication changes reduced but did not eliminate nightmares. A trial of quetiapine did not affect the hallucinations. Over the next six months, switching the sertraline to venlafaxine up to a dose of 300 mg daily did not significantly change his condition. The antidepressant was switched back to sertraline with the addition of bupropion 150 mg twice daily. Over the next year, the combination of sertraline 200 mg daily, bupropion 150 mg twice daily, and prazosin 5 mg at bedtime made only a mild impact upon his anxiety, nightmares, and psychosis. He was unable to attend recommended psychotherapy due to recurrent panic attacks. He began self-medication with alcohol consuming 3-4 drinks per day.

Two years after his initial evaluation, the veteran reported a fear he would act on his irritability/anger and harm someone at work. He said he did not plan to return to work. He reported daily auditory hallucinations and intermittent paranoia. Panic attacks occurred two to three times per week, even when he did not leave his home. Insomnia was a prominent complaint due to both initial insomnia and nightmares. He reported hopelessness and despair and didn't believe that any treatment could help him. He had stopped taking medication, but was willing to try a different medication if it might help. Tizanidine was started and titrated up to 4 mg at bedtime along with aripiprazole (15 mg at bedtime) and clonazepam (0.5 mg daily).

After two months, he was mildly improved. He had not experienced any panic attacks for the preceding two months. His mood and energy were somewhat better. Alcohol use was decreased to one to two beers per week with no binge use. However, other signs and symptoms of Major Depression, PTSD, and Psychosis NOS were unchanged. Tizanidine was increased to 8 mg at bedtime.

Two months later, he was markedly improved. He reported feeling less anxious during the day and was able to leave his house without panic attacks. He said his energy had improved. The nightmares were much less frequent and his sleep was improved. The hallucinations had stopped. Intrusive thoughts of combat or the Iraq war were both less frequent and when they occurred, less bothersome to him. He had not drunk any alcohol in the preceding two months. He disclosed he had stopped taking medications other than tizanidine approximately two to three months earlier. Due to the timing of medication refills, he continued taking tizanidine 8 mg at bedtime, but did not order refills of the sertraline, clonazepam, and aripiprazole. He admitted feeling ambivalent about taking psychiatric medication and said that was why he did not take the other three medications. He asked for a psychotherapy referral, saying his anxiety was at the point where he could comfortably drive himself to a weekly therapy appointment. He said his condition was the best he had felt since leaving military service three years earlier.

The improvement in this veteran's symptoms is remarkable considering he stopped his antidepressant, antipsychotic, and benzodiazepine after starting tizanidine.

### Case Summary 3:

The subject was a thirty-four year old Army veteran who served one year in Iraq and suffered from multiple psychiatric disorders. In Iraq, he had experienced near-daily firefights with anti-coalition forces in Iraq. During one such incident, he witnessed two of his fellow soldiers receive life-threatening wounds when a rocket-propelled grenade struck both soldiers inside their military vehicle. At significant personal risk, he drove his vehicle to a nearby hill to personally direct the medical evacuation helicopter toward the wounded soldiers. In addition to many additional combat experiences, he received numerous explosive blasts that left him feeling dazed or stunned.

The veteran was evaluated at multiple clinics at a veteran administration medical center, including psychiatry, neurology, neuropsychology, and speech pathology. The consensus diagnostic opinion was PTSD, Bipolar Disorder, Psychotic Disorder Not Otherwise Specified, Traumatic Brain Injury (TBI), Complex Migraine, Essential Tremor, and Cognitive Disorder due to TBI.

He was followed for more than two years for these conditions and treated with medications commonly known to be effective. These medications included sertraline (250 mg daily), divalproex sodium (2500 mg daily), quetiapine (100 mg twice daily and 400 mg at bedtime), amitriptyline (25 mg at bedtime), and tramadol (50 mg as needed for severe headache). None of these medications significantly reduced his signs or symptoms. A trial of tizanidine was started and the dose was titrated up to 8 mg at bedtime.

After four weeks taking tizanidine 8 mg at bedtime, he reported a number of beneficial effects, such as a substantial reduction in nightmares overall with a complete absence of combat-related nightmares. His sleep was significantly improved as a result of fewer nightmares, and he experienced a substantial decrease in daytime anxiety. He said he was able to engage in social situations that would have been impossible just two months earlier. He elaborated by noting his peak anxiety was not as high as it was prior to starting tizanidine. The veteran noted he found it easier to concentrate during the day and his attention was improved. The tremor was improved, both at rest and with action. The hallucinations were greatly reduced to the point that he no longer experienced vivid life-like hallucinations, and the residual hallucinations were an occasional voice that did not occur every day. Remarkably, the veteran said he had stopped the daily dose of quetiapine over the preceding four weeks. He noted the improvement in hallucinations despite decreasing the dose of quetiapine, an antipsychotic medication, by 33%. An added benefit to the reduced quetiapine dose was improved alertness during the day.

### Case Summary 4:

Male subject was admitted to inpatient psychiatry for severe anxiety, insomnia, auditory hallucinations, hypervigilance, irritability, and thoughts of harming others. These symptoms started during his combat tour in Iraq and worsened when he was home on leave midway through a twelve month combat deployment. He coped with these symptoms by drinking excessive amounts of alcohol. He came to the hospital for assistance and was admitted to the inpatient psychiatry unit.

Subject denied prior psychiatric history or head injury before his service in Iraq. Approximately four months prior to hospital admission, he sustained a blast injury from a large roadside bomb in Iraq, characterized by a concussion with loss of consciousness, partial jaw fracture, ruptured tympanic membrane, a bruised/dislocated shoulder, whiplash, and a lower-back injury. The bomb explosion threw him from the truck he was driving. After regaining consciousness, he was initially confused then experienced drowsiness prior to and during his evacuation to a military hospital. There is a known correlation with ruptured tympanic membranes and risk of traumatic brain injury, and nearly all diagnostic criteria for TBI include loss of consciousness or post-injury confusion. After the blast, he developed daily severe headaches and dizziness.

A MRI study of the brain was performed and interpreted by the neuroradiology staff as negative for any visible intracranial pathology. After hospital admission, subject was evaluated further and reported additional complaints. He reported poor short-term memory, intrusive memories of combat scenarios in Iraq and of the explosion that injured him, mistrustfulness of other people or crowds of people, fearfulness of debris or trash on or beside the road, and angry outbursts with little provocation. He also reported regular panic attacks.

Subject reported that tizanidine 4 mg at bedtime reduced his nightmares and improved the quality of his sleep. He also reported feeling less irritable and less anxious during the day.

Psychological Testing Results: The diagnosis of PTSD was made with the criteria in the American Psychiatric Association Diagnostic and Statistical Manual, Fourth Edition, (DSM-IV) based upon symptoms reported by the subject. These diagnoses were confirmed by psychological and neuropsychological testing administered approximately one week after he left the inpatient psychiatry unit. The psychological testing included BDI-II, DTS (a measure of PTSD), and MMPI-2 (a standard measure of personality function that also is useful in diagnosing common psychiatric conditions). These psychological tests revealed a BDI-II score of 38, a score that is consistent with severe depression. On the BDI-II, the subject reported significant distress from the following emotional symptoms: sadness, pessimism, indecisiveness, irritability, loss of energy and interest in people and activities, concentration difficulty, and changes in appetite. On the DTS, the subject's results were a score of 134, a result consistent with PTSD. The MMPI-2 results supported diagnoses of PTSD with perceptual disturbances. The MMPI-2 results were considered clinically informative and relevant despite elevated validity scales (L scale T score=52, F scale T score=101, K scale T score=30) because a similar pattern is seen frequently among persons with untreated PTSD. The clinical scales revealed elevations on nine of ten scales, an unusual pattern (Welsh code=8**76*123″490′-/5). Subject's profile included elevation on scales 8 and 7, and the PS and PK scales. PS and PK are MMPI scales specific for PTSD. PS=Schlenger scale; PK=Keane scale. Elevated PS identifies persons with PTSD compared to controls. Elevated PK identifies persons with PTSD compared to psychiatric patients with other diagnoses. The PS and PK scales are elevated commonly among persons with PTSD. Other symptoms typically reported by subjects with the MMPI-2 profile similar to the present individual include: suffering from chronic stress, extreme agitation, brooding, dysphoria, ruminative doubts and difficulty with concentration and thinking. The elevated scale six and related fears sub-scale suggests that subject was feeling an unusually high degree of suspiciousness and mistrustfulness of other persons and/or objects in his environment, perhaps experienced as feeling others are persecuting him. These scores could also reflect a fear of objects or situations in his every day life.

Additional psychological test results and clinical presentation were most consistent with PTSD, Cognitive Disorder of fronto-subcortical regions due to TBI, Major Depression, and Panic Disorder.

Pharmacologic Treatment and Response: While on the psychiatry unit, he was treated with sertraline for PTSD and nortriptyline for headaches and depression. Five days after his hospital discharge, he reported improvements regarding hallucinations and paranoia but persistent sleep problems and nightmares. He also reported reduced but continued self-medication with alcohol. He was started on tizanidine 4 mg at bedtime. Approximately three weeks after starting tizanidine, subject reported his nightmares were somewhat improved.

This case summary illustrates that tizanidine reduced nightmares and improved sleep for a subject diagnosed with PTSD, Traumatic Brain Injury, and Panic Disorder.

### Case Summary 5:

Subject developed psychiatric difficulty during his military service. He was presented to an outpatient clinic for psychiatric care. The initial psychiatric evaluation found signs and symptoms of PTSD. The subject reported exposure to a traumatic event and responded with helplessness and honor. The traumatic event reported by this subject was re-experienced in the following ways: (1) recurrent, intrusive recollections of the traumatic event and (2) nightmares. The subject attempted to avoid reminders of the traumatic event and/or experiences and had decreased interest in usual activities. There was evidence of increased emotional arousal that developed after the traumatic event marked by insomnia, irritability or angry outbursts, difficulty concentrating, hyper vigilance, and increased startle response. In addition to evidence of PTSD, the evaluation discovered signs and symptoms of a mood disorder as follows: depressed mood most every day; insomnia or hypersomnia nearly every day; diminished energy and interest in usual activities; decreased concentration; decreased appetite; and psychomotor retardation or agitation (as observed by others). The signs and symptoms above met the DSM-IV criteria for PTSD and major depression. Subject said he was exposed to regular military combat in Iraq, including one episode of intense fighting that resulted in his discovering the dead body of a close friend in his military unit.

Since his release from military duty, he had been treated with sertraline with poor results. He denied any psychiatric problems or head injury prior to military service. He did not receive a head injury while in the military. He reported drinking several times per week. His presentation was primarily marked by an inability to function effectively at work, at home, or interpersonally with his family. The initial diagnostic impression was major depression and PTSD so the sertraline was increased. Over the next twelve months, the subject showed only a modest response to various medication changes, including increasing the sertraline to 200 mg per day and the addition of a mood stabilizer (sodium divalproex) and an atypical antipsychotic (aripiprazole). He continued to have prominent signs and symptoms of PTSD, major depression, and developed recurrent panic attacks.

After approximately one year, subject agreed to a trial of tizanidine as an addition to sertraline. During a phone call several weeks after starting tizanidine, subject reported a dose of tizanidine 2 mg at bedtime resulted in no change to nightmares and caused no side effects or other adverse effects. He agreed to increase the dose to 4 mg at bedtime and follow-up in four weeks. Four weeks later, he reported he was not doing well. He had experienced a panic attack at work the prior week, and was fearful others at work were watching him or are making fun of him. This fear caused him to worry he might act on irritability/anger and harm someone at work. The subject also reported at this visit he had been experiencing daily auditory hallucinations, daily paranoia, nightmares 3-4 times per week, and panic attacks 2-3 times per week. The description of how he experienced a panic attack and the recurrent panic attacks met the DSM-IV criteria for panic disorder. He said his insomnia was pronounced and was caused by both anxiety and nightmares. It was recommended to the veteran and his employer that he not return to work out of concern his irritability and anxiety might cause him to harm others. An atypical antipsychotic medication, aripiprazole, was added to his medications.

Eight weeks later, the subject said he noticed limited improvement with tizanidine 4 mg at bedtime and he reported an improvement in his mood and energy level. He had been free from panic attacks for several weeks now. Alcohol use was decreased to only one to two drinks per week with no binge drinking. However, the remainder of his PTSD and depressive symptoms were unchanged, and nightmares remained a significant problem. He reported daily or near-daily disturbing recurrent thoughts about Iraq. He experienced episodes during the day when he was unsure if he was hallucinating or was lost in memories of Iraq. There were no side-effects from tizanidine. The tizanidine dose was increased to 8 mg at bedtime.

Eight weeks after increasing the tizanidine to 8 mg at bedtime, subject reported substantial improvement. He noted this improvement despite not taking sertraline and aripiprazole for at least several weeks and perhaps up to two months. The nightmares were improved and panic attacks continued to be absent. He reported feeling less anxious and more energetic during the day. His alcohol consumption had decreased to less than once per month. He said he and his wife both noticed positive changes in their relationship and he felt better able to manage the usual stress of parenting his young children.

At a primary care physician appointment one month later, subject reported decreased nightmares, less anxiety, and improved depression. He reported less nervousness in public places. He reported he still enjoyed normal activities and his appetite was increased. He denied having any hallucinations.

This case summary illustrates that tizanidine reduced nightmares, improved sleep, reduced auditory hallucinations, relieved panic attacks, and improved depressive symptoms in a subject diagnosed with PTSD, a psychotic disorder, NOS, a panic Disorder and Major Depression.

### Case Summary 6:

Subject retired from the military after a 20-year career during which he experienced combat action in two different wars. He received a thorough psychological evaluation, including an interview and standardized psychological tests: the Combat Exposure Scale (CES), Mississippi Scale for Combat-Related PTSD, the Davidson Trauma Scale (DTS), the Alcohol Use Disorders Identification Test (AUDIT), the Beck Depression Inventory (BDI-II), the Minnesota Multiphasic Personality Inventory-2 (MMPI-2), the Personality Assessment Inventory (PAI), and the Clinician Administered PTSD Scale (CAPS) for the DSM-IV. The result of the interviews and psychological tests were believed valid and interpretable. Subject's scores on both the Mississippi and DTS tests exceeded the clinical cut-offs for a diagnosis of PTSD. He endorsed light to moderate combat exposure on the CES. The AUDIT test result suggested a potential for hazardous or harmful alcohol consumption. Scores on the BDI-II indicated a possible moderate depressive disorder without any plan or intent to harm himself. Subject's validity profiles for both personality tests (MMPI-2 and PAI) were consistent with persons who report severe psychological distress, a diagnosis of PTSD, or both. These scores indicated he took a candid approach to the tests with valid and interpretable profiles.

MMPI-2 results were consistent with those reported by other individuals diagnosed with PTSD. Elevations on the PK and PS scales, both PTSD specific measures, were consistent with a diagnosis of PTSD. Additional information from the MMPI-2 suggested significant anxiety and depressive symptoms, disrupted concentration, social avoidance and estrangement from others, and irritability. MMPI-2 testing also indicated subject felt misunderstood by others, has an overly anxious or even mildly paranoid view of other persons and the world in general. Results further suggested physical health concerns, relationship distress, and the potential to feel emotionally out of control. Individuals with test results similar to subject have been described as negativistic or pessimistic.

The PAI results also indicated a diagnosis of PTSD. The PAI results were similar to those reported by persons with distress and serious impairment following a traumatic experience. The PAI results also suggested subject was likely to report nightmares, sudden anxiety reactions, and feelings of being irreversibly changed by the traumatic event. Although his responses suggested anxiety toward specific situations, clinical questioning did not support a phobia diagnosis, nor did it support hallucinations. His unusual sensory experiences identified on testing were most consistent with flashbacks related to PTSD. The testing data also suggested subject was aware of his excessive substance use.

The psychiatric interview identified a number of specific traumatic experiences, including exposure to wounded or dead friendly and enemy military personnel and destroyed military equipment. He endorsed a constant fear of being attacked in a manner similar to the devastation he saw in war. During later military experiences, he was attacked by indirect fire and feared for his life. Subject reported numerous signs and symptoms of PTSD. These included intrusive and distressing memories about his experiences in Iraq on a weekly basis that he was unable to dismiss; using alcohol in an effort to cope with these memories; and distressing dreams related to his war experiences several times a week. He reportedly awoke from these dreams confused and anxious, with significant trouble returning to sleep. He also experienced weekly dissociative flashbacks. He also had occasional visual hallucinations of wartime memories. Subject also reported psychological distress and physiological reactivity when exposed to events or situations that reminded him of his combat experiences, and attempted to avoid those situations (news reports, interpersonal exchanges about the war). He reported using alcohol and marijuana to avoid emotions related to PTSD. Subject said he had moderate difficulty recalling parts of his wartime experiences. He said he had lost interest in activities he previously enjoyed (e.g., playing or watching sports), and the only activity he still enjoyed was his time with his children. He reported he felt estranged from other people except his children, and had trouble expressing himself emotionally. Based on these and other complaints, he was diagnosed with Chronic PTSD, Alcohol Abuse, and Marijuana Abuse, and referred to psychiatry for further management.

He was treated with sertraline up to 200 mg per day with only modest improvement in his PTSD signs and symptoms. At an outpatient visit after approximately one year of treatment, he reported the following signs and symptoms of PTSD that had not responded to sertraline 200 mg per day: insomnia (both initial and middle insomnia); nightmares; flashbacks (approximately one per month); irritability; social withdrawal; not able to maintain social or romantic relationships; recurrent thoughts of war experiences; and anger when reminded of wartime experiences. The overall psychiatric impression was of PTSD that had not yet responded to a high dose of sertraline. Treatment with tizanidine 4 mg at bedtime was added to the existing sertraline.

Approximately four weeks later, subject reported he was taking tizanidine 4 mg at bedtime. Since starting tizanidine, he noticed a reduction in his nightmares, fewer night sweats, and while it had not increased his sleep time, he felt more rested the next day. He said he thought his overall sleep quality had improved since starting tizanidine.

This case summary illustrates that tizanidine reduced nightmares, improved sleep quality, and improved daytime energy in a patient diagnosed with PTSD, Marijuana Abuse, and Alcohol Abuse.

### Example 2

This example provides three case summaries that illustrate the effectiveness of switching from once to twice-daily dosing of tizanidine, typically as 8 mg at bedtime to 4 mg in the morning and 4 mg at bedtime.

### Case Summary 7:

The subject was a veteran of military service in Iraq who presented signs and symptoms of a number of disorders including: PTSD; Bipolar Disorder; Psychotic Disorder, Not Otherwise Specified; Traumatic Brain Injury; and Migraine Headaches. In addition to these diagnoses, his symptoms are highly suggestive of either or both of partial-complex seizures or dissociative episodes. Subject reported he had not experienced any psychiatric problems or head injury prior to his combat experiences. His emotional and physical problems started during his combat tour and have continued since leaving active military service. He was exposed to significant combat action, including witnessing soldiers in his unit severely injured by enemy fire and being attacked by roadside bombs on multiple occasions. Subject reported he was exposed to several improvised explosive devices in Iraq. The most severe explosion involved at least one, possibly two, large artillery rounds command-detonated beneath his non-armored military vehicle. He reported a loss of consciousness of less than five minutes after this explosion, followed by the onset of intense headaches, nausea, and tinnitus. He denied any history of headaches prior to his Iraq military service. He also reported the onset of a bilateral hand tremor that interfered with his dexterity that developed during or after his military service in Iraq. A recent MRI scan was notable for global atrophy greater than expected for his age and medical problems. He also reported problems with his legs feeling “twitchy” at night. He was unsure if it interfered with sleep, but said it impaired his ability to fall asleep. He denied alcohol or illicit drug use while under care at this facility or a past history of alcohol or drug use.

The subject reported a history of manic and depressive episodes that started after his return to civilian life. These episodes were marked by racing thoughts, not being able to talk fast enough to express his thoughts, accomplishing more activity than usual, decreased need for sleep, engaging in impulsive and reckless behavior, and spending large sums of money. During these episodes, his mood was described as markedly elevated from its usual level. The mood symptoms included depressive episodes as well. He described episodes of depressed mood, loss of interest, fatigue, decreased energy, insomnia despite taking quetiapine 500 mg at bedtime, poor personal hygiene, thoughts of suicide, and hopelessness. These symptoms were only somewhat responsive to prior medication trials. His family history was negative for depression or bipolar disorder.

Even when his mood was neither depressed nor elevated, he reported a sleep disturbance characterized by difficulty both initiating and maintaining sleep. He experienced war-related nightmares almost every night.

He also reported a number of PTSD symptoms such as marked anxiety, being easily startled, significant irritability, and hypervigilance. He was uncomfortable in crowds, avoided television shows about combat, and had regular intrusive thoughts or memories of the war. Subject avoided going out in public, and when he did, he felt he must continually monitor his surroundings out of fear he might be attacked. He had difficulty falling asleep and could not remain asleep due to nightmares. During the day, he was easily irritated and could be provoked into episodes of rage with little provocation. He felt irritable or angry when exposed to reminders of the current war.

Subject was referred for neuropsychological testing. The neuropsychology referral was initiated after routine TBI screening indicated lower than expected cognitive function on a screening battery consisting of several neuropsychological tests.

The overall conclusion from neuropsychological testing was impaired attention, executive functioning, manual dexterity, and verbal learning; the conclusion also found psychiatric diagnoses of PTSD, Bipolar Disorder, and Psychotic Disorder, NOS. The neuropsychological impairments were judged to be in excess of those expected for the psychiatric problems of PTSD, Bipolar Disorder, and Psychosis NOS. The deficit pattern suggested an anatomic location in the left mesial temporal lobe, most likely caused by head trauma.

Subject was treated with a variety of medications for approximately one year. These medications included quetiapine, aripiprazole, sertraline, divalproex, trazodone, and hydroxyzine. He showed only modest improvement on these medications despite their being prescribed at doses close to the maximum recommended dose on the package labeling.

After approximately 18 months of outpatient treatment, subject developed a severe depressive episode.

At the time of admission, he described a full range of depressive symptoms including hopelessness and suicidal thoughts. He also reported prior symptoms consistent with manic episodes, including at least one occasion lasting a week with racing thoughts, decreased need for sleep, spending sprees, and engagement of dangerous behaviors not typical for him. He also reported both auditory and visual hallucinations with the PTSD. He denied alcohol or drug use. After a brief inpatient stay, he was returned to outpatient care with a plan to be admitted to a long-term inpatient PTSD treatment unit.

Upon return from a veteran hospital inpatient PTSD program four months later, subject reported improved PTSD symptoms. Four months after discharge from the inpatient PTSD program, subject was seen for a routine outpatient visit. He reported that while the recent inpatient program somewhat reduced his anxiety where he could begin part-time work, he noted the onset of panic attacks. The panic attacks were characterized by an abrupt onset of intense anxiety accompanied by several physical symptoms: dizziness, palpitations, chest tightness or difficulty breathing, and feeling lightheaded.

Subject also reported a recent increase in visual hallucinations and he sometimes had olfactory hallucinations to accompany the visual hallucinations. The recent addition of topiramate did not reduce the frequency of these combined visual-olfactory hallucinations. He said his spouse reported that he was not responsive to verbal stimuli for several minutes after the combined visual-olfactory hallucinations began. Separately, he continued to have visual hallucinations that were not accompanied by olfactory hallucinations or altered level of consciousness. He was continued on aripiprazole, quetiapine, sertraline, and trazodone. Treatment with tizanidine was started for the nightmares and poor sleep.

Four weeks later, subject reported a significant change in his condition. He said that since he started taking tizanidine at a dose of 8 mg at bedtime, he experienced the following: greatly reduced nightmares overall; a complete absence of combat-related nightmares; significantly less anxiety during the day; improved tremor; fewer hallucinations. While this clinical improvement in only four weeks was remarkable, subject also reported he had self-directed some changes to his medications. He eliminated three of the scheduled four doses of quetiapine and reported the sedating side-effect of quetiapine was no longer present. Overall, subject reported significant satisfaction with tizanidine. He was switched from tizanidine 8 mg at bedtime to tizanidine 6 mg in the morning and 8 mg at bedtime. Two months later, subject reported he continued to feel “pretty good overall.” He said his prior difficulties with anxiety, mood, and sleep continued to be greatly improved with tizanidine. He reported specifically on changes in his current signs and symptoms after switching to the twice-daily tizanidine dosing. This dose switch resulted in greater reduction to his daytime anxiety with no change to the beneficial impact on nightmares and sleep quality. The medication switch improved his condition to where he was able to stop the aripiprazole. He continued on the sertraline, divalproex, and trazodone for initial insomnia. The tizanidine was switched to 4 mg in the morning and 4 mg at bedtime.

One month later, subject reported on the impact of switching the tizanidine from 8 mg at bedtime to 4 mg twice daily while continuing without change the sertraline, divalproex, and trazodone: continued to have no nightmares; continued to have restful sleep; no change to daytime anxiety; no change in daytime cognition; no changes in mood; and definite reduction in his desire to smoke cigarettes. He had previously failed at an attempt to stop smoking while taking tizanidine. After the switch to tizanidine 4 mg twice daily, subject noted a reduced desire to smoke.

### Case Summary 8:

Subject presented to the outpatient clinic requesting evaluation and treatment for anxiety symptoms. His symptoms began while he was involved in combat. He related the onset of his anxiety to witnessing several combat deaths among soldiers in his platoon. His anxiety initially began as the abrupt and unexpected onset of intense anxiety accompanied by chest pain, palpitations, dyspnea, and diaphoresis. He was treated with Venlafaxine up to 150 mg daily and developed akathisia. He did not receive further treatment until after leaving the combat zone. While in a combat zone, subject was exposed to several significant blast injuries. At least one such injury caused him to lose consciousness. A different explosion resulted in his suffering a ruptured tympanic membrane (TM). After the latter injury, he experienced problems with thinking and judgment the next day.

After discharge from the Army, subject reported the onset of multiple PTSD signs and symptoms as: nightmares (1-4 times per week); intrusive and bothersome recollections of wartime experiences; avoidance of war reminders (“I don't watch TV any more”); feeling estranged from people except for his spouse (“it's like I can't relate to people any more”); unsure about his future; hypervigilant (“my wife says hello when she comes home so I'm not caught off guard”); startled very easily (“I can't tolerate anyone tapping me on the back unless I know they're there beforehand”); and had significant trouble with insomnia.

He reported self-medication with alcohol, up to 9 drinks per session, in binge use of alcohol when he felt overly anxious. He did not drink every day. He was a non-smoker and did not use illicit drugs. Citalopram 20 mg daily was started two months prior to his initial evaluation with the only benefit of a slight reduction in irritability. He denied any history of psychiatric illness, head trauma, or family history of psychiatric illness. Medical complaints at initial assessment included headaches, decreased hearing, tinnitus, and decreased sense of both smell and taste, all of which started during or shortly after he left the combat zone. He said he was diagnosed with TBI by the military subsequent to the blast injuries described above. He denied any episodes of lost time, ending up in unexpected physical locations, or inattentiveness. There was no known history of seizures. He reported ongoing left ear pain with mild high frequency sounds. This symptom developed after his combat tour. Previous evaluation performed by the military included a MRI, EEG and consultation with Audiology and Neurology, all of which were unrevealing.

Initial physical examination revealed neurologic difficulties of poor dynamic balance, with the tandem gait also impaired. There was not a loss of balance but subject did demonstrate difficulty performing the gait and balance tasks. He was referred for a full neuropsychological examination. The subject's results were consistent with PTSD. The malingering index, suicide potential index, and violence potential index were all within normal limits, as were indices related to antisocial symptoms and anger. Subject denied hallucinations or delusions that could indicate a primary psychotic disorder. On cognitive testing, his intellectual functioning was within the average range with a six-point differential between verbal and performance IQ scores. The neuropsychologist noted some indications of limited effort. It was suggested that under-treated PTSD was the cause of poor motivation. The initial treatment was an increase of citalopram to 40 mg daily. Two months later, subject reported no significant change to his panic attacks. He also reported the new onset of agoraphobia and seeing “shadows” in his peripheral vision. He was switched to sertraline at a dose of 150 mg per day. Two months later, he reported his anxiety symptoms had responded well to sertraline, and he was able to go out in public with less anxiety. Irritability, however, was not improved.

Subject was started on tizanidine up to 8 mg at bedtime for nightmares. He also was issued a device to assist with memory problems. A rating scale for common cognitive problems was administered by speech pathology prior to issuing him a personal digital assistant as a memory assist device. His cognitive function improved over the following weeks.

With the improved cognitive function, the subject was referred to psychology for psychotherapy. He continued to take sertraline and tizanidine. At a psychology evaluation session, subject completed two self-report instruments of PTSD and depressive symptoms to establish a baseline indication of symptom severity. The test results showed severe PTSD (PTSD Checklist score=67) and moderate to severe depression (Beck Depression Inventory II=30). The subject was seen in sleep disorder clinic the following month. He reported horribly vivid dreams that started during his second combat tour. He was initially treated with zolpidem, but that medication did not improve the nightmares. Subject reported a significant improvement in “feeling better the next day” only after he started the tizanidine. He had not enacted a dream for several months. Subject was then switched from tizanidine 8 mg at bedtime to 4 mg twice daily. Two weeks later, he reported that since switching from tizanidine 8 mg at bedtime to 4 mg two times daily, he noticed the following: no change in sleep (e.g., the 4 mg dose still helped his sleep and eliminates nightmares); new onset mild decrease in energy that was gradually improving; new onset of less irritability during the day; and new onset of reduced hypervigilance during the day. This case study illustrates tizanidine improves cognition, sleep and reduces nightmares.

### Case Summary 9:

Subject was referred for mental health evaluation by his primary care physician. At the time of his initial evaluation, subject had been awarded a partial disability for PTSD by the Veterans Benefits Administration. He reported his first and second combat tours were in two different conflicts separated by more than ten years. He reported regular intrusive memories and nightmares of war. The subject did witness and experience traumatic events that included actual or threatened death or serious injury to himself and others, and he re-experiences these through nightmares and intrusive thoughts. He avoided triggers that remind him of the events. He stated he was less interested in social activities, felt distant from others, had a sleep disturbance, was irritable, hypervigilant and easily startled. These problems interfered with interaction with others and caused distress. He met the DSM-IV criteria for PTSD.

Subject's presentation also included a head injury sustained in his first combat tour. During that tour, he was rendered unconscious by incoming artillery fire.

After approximately one year of being followed in the psychiatry clinic, he became more willing to try medication and agreed to a trial of tizanidine. Two months later, he reported a definite improvement with this medication: the headaches were much less intense although still present. He reported less anxiety but still startled easily and was hypervigilant. His mood and energy were improved, sleep was better, and he felt more relaxed overall. His cognitive symptoms were unchanged. Subject tried various combinations of tizanidine 4 mg tablets and found the best result from 4 mg twice daily. He had tried taking two tablets (8 mg) at bedtime and preferred the 4 mg twice daily combination. He said there was a clear superiority of twice-daily compared to once-daily dosing even if the total milligrams of medication remained unchanged.

This case summary demonstrates results from switching once to twice-daily dosing and the value of as-needed administration of the second daily dose. Also illustrated is the incremental benefit of the second daily dose, even if the total of two daily doses is the same as a single once-daily dose.

### Example 3

This example provides a case summary that illustrates the effectiveness of using tizanidine with methylphenidate to treat PTSD from assault and not military action.

### Case Summary 10

Subject was a African-American female veteran of the United States military who was diagnosed with PTSD. She was medically separated from the military for depression and PTSD. A psychological evaluation in 2007 documented several psychiatric symptoms. The developmental history also was notable for mild to moderate physical abuse from extended family members. She graduated from high school with an average GPA and was socially popular with peers. She joined the military to escape her abusive family members. Her military career was uneventful. She was an honor graduate in basic training. She successfully completed a succession of military training courses and assumed more roles within her military unit. She steadily advanced in responsibility and military rank. She had served in the military for more than ten years in a logistics role. Her military career was characterized by several deployments in support of special operations forces. The patient was an assault victim. She was abducted, held, and physically assaulted with resulting injuries as broken mandible (jawbone), hyphema, concussion, a dislocated tooth, and a separated shoulder.

A number of clinical questionnaires were used as part of the overall psychological assessment and revealed Major Depression—moderate—single episode, PTSD, and Alcohol Dependence—in sustained full remission.

Her past psychiatric history was remarkable for Attention-Deficit Hyperactivity Disorder (ADHD) that was diagnosed during her military service. She received medication trials of both bupropion and methylphenidate, neither of which were effective for her ADHD symptoms. Neuropsychological testing average results on measures of attention/concentration (WAIS-III: Working Memory Index=97) and information processing speed (WAIS-III: Processing Speed Index=81). Motor speed and simple sequencing was average (Trails A, T=46) and the more complicated Trails B score was mildly impaired (Trails B, T=37). Response inhibition was within the average range. She performed in the average range on tasks requiring selective attention and vigilance, without demonstrating impulsivity or perseveration. However, attention test performance declined with decreasing inter-stimuli intervals, suggesting a problem with cognitive switching. Language function tests demonstrated above average object naming and phonemic verbal fluency, but categorical verbal fluency was mildly to moderately impaired. On a psychological test, the PAI, her responses endorsed a large number of symptoms, a pattern that is observed commonly among persons with PTSD. She performed well in a test designed to test motivation and effort.

The neuropsychologist concluded that the patient's overall responses suggested a local brain dysfunction in the mesial temporal lobes, left side worse than right. The neuropsychologist noted that the observed deficits were in excess of those expected for a patient with a history of PTSD, Major Depression, and ADHD. At her initial evaluation at this facility, she was started on sustained-release venlafaxine and the dose was gradually increased to 225 mg per day. She was referred for neuropsychological testing and to the neurology clinic. The patient informed the neurologist that she had been assaulted several years earlier when her head was hit against the ground repeatedly and she lost consciousness for at least 10 minutes. She reported the onset of PTSD symptoms after the assault. She also reported the onset of vision, memory, sleep and pain symptoms. She experienced nightmares and night sweats most nights. She could fall asleep initially, but when she awoke she was unable to go back to sleep. Last, she reported having difficulty with pain which most affects her shoulders and knees. She suffered from severe headaches soon after her head trauma, but explained that they had gradually become less frequent and less painful. Now, she had some difficulty with tension headaches brought on by stress and anxiety, but they respond well to medication, and were not debilitating.

The mood and anxiety symptoms were only modestly improved with venlafaxine sustained-release 225 mg per day and zolpidem 10 mg at bedtime for insomnia. Given the prior history of ADHD, methylphenidate was cautiously added to the venlafaxine. The methylphenidate-venlafaxine combination brought a marked improvement in her mood and anxiety symptoms. She was referred to a specialty inpatient program for PTSD. During the inpatient program, the antidepressant was switched from venlafaxine to duloxetine. She did not believe the duloxetine was helpful and asked to try methylphenidate as monotherapy. After several weeks with taking only methylphenidate, she reported improved mood and anxiety symptoms.

She believed methylphenidate was helpful for her mood, anxiety, and cognition and wished to continue that medication. The switch made several months earlier to short-acting methylphenidate 10 mg three times daily brought additional improvements in her mood, anxiety, and cognitive symptoms. Stopping the antidepressant medication at the time of increasing the methylphenidate did not change her mood or anxiety symptoms.

After one year of treatment, she reported middle insomnia due to nightmares that occurred three to five times weekly. She was started on tizanidine 4 mg twice daily, and after four weeks, returned to clinic to report it had been extremely helpful. She reported greatly improved sleep with an absence of nightmares. She had been taking zolpidem several nights per week to help with sleep and had not stopped using it. She said “I can fall asleep and the tizanidine lets me stay asleep. Since I don't wake up from nightmares, I don't need the zolpidem to stay asleep.” She noted less daytime anxiety when taking tizanidine 4 mg twice daily but was taking it only at night because the tizanidine caused mild sedation. She said that better sleep has helped her mood, anxiety, and cognition.

This case summary demonstrates the effectiveness of using tizanidine and methylphenidate (a stimulant medication) to treat PTSD and/or TBI without the administration of an antidepressant medication. This case summary also illustrates the effectiveness of using tizanidine to treat PTSD resulting from assault, not military action.

### Example 4

Based upon the teachings herein, tizanidine or a similar substance that is known or suspected to have activity at the imidazoline receptor can be evaluated in a clinical trial to determine the effective concentration and dosage regimen to treat an identified psychiatric disorder. In one instance of a clinical trial, tizanidine is tested against a placebo (such as a sugar pill). A purpose of this trial would be to establish the effectiveness of tizanidine when neither the physician nor the test subject knows whether tizanidine or the placebo has been administered. This trial is designed to include 20 test subjects each in the control (sugar pill) and test (tizanidine) study groups. The placebo and test pills are physically identical so neither the study subject nor study physician can determine whether the subject is taking placebo or tizanidine. After the study experimental protocol and informed consent forms are approved by the appropriate institutional review board, study subjects are recruited. After giving informed consent to participate in a research study, potential study subjects complete standardized psychiatric rating instruments prior to trial enrollment; these instruments include tests intended to detect and measure the severity of psychiatric disorders such as PTSD (PTSD Checklist, Clinician-Administered PTSD Scale, or Davidson Trauma Scale), Major Depression (Beck Depression Inventory, Hamilton Depression Rating Scale), psychosis (Brief Psychiatric Rating Scale), or anxiety (Hamilton Anxiety Rating Scale). In some cases, these tests may be used to measure symptoms where a non-psychiatric disorder can cause psychiatric symptoms; one example of this situation could be depression that occurs after a TBI. In still other cases, the study subject's self-reported symptoms and the change in those symptoms is a valid experimental measurement. In order to adequately control for possible confounding factors, the potential subjects are asked to answer questions about past psychiatric diagnoses and treatments, current medications, current medical problems, and current/recent substance use patterns. Once the study investigators determine that the potential subject has the disorder being studied (e.g., PTSD) and does not have a possible confounding disorder (e.g., untreated bipolar disorder; daily consumption of more than four alcoholic drinks per day; untreated hypothyroidism), that subject will be randomly assigned to either the placebo or test group. In this study design, the members of the groups and study physicians do not know which group the specific subjects belong (e.g., the pharmacist is the only one that knows if the study subject received tizanidine or placebo), thereby allowing the study subject and study physician to record only objective facts and avoid a potential reporting bias with respect to ongoing study measures. These ongoing measures include the study subject's reported change in signs or symptoms being studied, standardized tests (e.g., PTSD Checklist or other tests) intended to capture information about a specific diagnosis, or standardized tests intended to capture overall clinical improvement. These measures are given at various intervals, including prior to the initial medication or placebo dose, and again after two, four, six, and eight weeks in the study. In a particular example, the subject begins the study taking one placebo tablet twice daily or one tizanidine 4 mg tablet twice daily. In this same particular example, the study physician gradually increases the prescribed tablets from one in the morning and one in the evening up to a maximum of four tablets each in the morning and evening. The tablets are increased on a basis of no more than one additional tablet each in the morning and evening per week, therefore taking four weeks to reach the maximum dosage of four tablets each in the morning and evening. After a study period of six to eight weeks, test subjects are taken off their study intervention and the study physician reviews test scores by the study category (drug vs. placebo) and dosage (number of tablets in the morning and evening). In this particular example, the study results will show how the signs and symptoms being studied changed over time with gradually increasing doses of tizanidine (e.g., 4, 8, 12, or 16 mg taken twice daily compared to sugar pills taken twice daily). The study physician performs statistical analyses to determine if the study hypotheses are statistically significant. In this example of only one study that could be performed, hypotheses can include: (1) “the administration of tizanidine at a dose of at least 4 mg twice daily provides better management of PTSD signs symptoms than a placebo, according to study subject self-reports”; (2) “administration of tizanidine at a dose of at least 4 mg twice daily provides better management of PTSD signs symptoms than a placebo, as indicated by at least a 20% improvement on the PTSD Checklist after 8 weeks of medication treatment”; (3) “increasing the tizanidine dose from 4 mg twice daily up to 16 mg twice daily will provide greater relief from signs and symptoms of PTSD”; and (4) “increasing the dose of tizanidine from 4 mg twice daily up to 16 mg twice daily will not change the frequency or severity of reported medication side-effects.” At least a 20% improvement, such as at least 25%, at least 30%, at least 40%, at least 50%, at least 60%, at least 70%, at least 80%, at least 90%, or at least 95%, measured by the clinical rating scale indicates that the tizanidine dosage may be used to treat the given psychiatric disorder.

In yet another example of a clinical trial to determine the optimal method for administering tizanidine for the relief of specific psychiatric signs or symptoms, tizanidine may be administered with another medication. Non-limiting examples of another medication includes antidepressant medication, antipsychotic medication, anxiolytic medication, stimulant medication, or mood stabilizing medication.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method of treating a sleep disorder or an anxiety disorder, comprising:
selecting a subject with a sleep disturbance or anxiety disorder in the absence of an underlying physical disorder; and
administering a therapeutically effective amount of tizanidine, thereby reducing or inhibiting a symptom of the sleep disorder or anxiety disorder.

2. The method of claim 1, wherein the sleep disturbance comprises nightmares.

3. The method of claim 1, wherein the sleep disturbance or anxiety disorder is associated with a neurological or psychiatric disorder.

4. The method of claim 3, wherein the neurological or psychiatric disorder comprises one or more of Post-Traumatic Stress Disorder, Parkinson's disease, a brain injury or a partial complex seizure disorder.

5. The method of claim 1, wherein administering a therapeutically effective amount of tizanidine comprises administering about 2 mg to about 20 mg of tizanidine.

6. The method of claim 1, wherein administering a therapeutically effective amount of tizanidine comprises administering about 4 mg of tizanidine.

7. The method of claim 5, wherein tizanidine is formulated as a time-release formulation which releases about 5% of the original dose per hour.

8. The method of claim 5, wherein tizanidine is formulated as a time-release formulation which releases about 8%, of the original dose per hour.

9. The method of claim 1, wherein administering a therapeutically effective amount of tizanidine comprises administering tizanidine daily.

10. The method of claim 1, wherein administering a therapeutically effective amount of tizanidine comprises administering tizanidine twice daily.

11. The method of claim 1, further comprising administering a therapeutically effective amount of an antidepressant, antipsychotic, mood stabilizer, stimulant, anticonvulsant, benzodiazepine, or a prodrug or active metabolite of any of these medications, or a combination thereof.

12. A method of treating a mood disorder or a perceptual disturbance, comprising:
selecting a subject with a mood disorder or perceptual disturbance; and
administering a therapeutically effective amount of tizanidine, thereby reducing or inhibiting a symptom of the mood disorder or perceptual disturbance.

13. The method of claim 12, wherein the mood disorder comprises a depressive disorder, an elevated mood, a manic mood, an irritable mood or a combination thereof.

14. The method of claim 12, wherein the perceptual disturbance comprises a visual disturbance, an auditory disturbance, an olfactory disturbance, a dissociative state or a combination thereof.

15. The method of claim 12, wherein administering a therapeutically effective amount of tizanidine comprises administering about 2 mg to about 20 mg of tizanidine.

16. The method of claim 12, wherein administering a therapeutically effective amount of tizanidine comprises administering about 4 mg of tizanidine.

17. The method of claim 15, wherein tizanidine is formulated as a time-release formulation which releases about 5% of the original dose per hour.

18. The method of claim 15, wherein tizanidine is formulated as a time-release formulation which releases about 8%, of the original dose per hour.

19. The method of claim 12, wherein administering a therapeutically effective amount of tizanidine comprises administering tizanidine daily.

20. The method of claim 12, wherein administering a therapeutically effective amount of tizanidine comprises administering tizanidine twice daily.

21. The method of claim 12, further comprising administering a therapeutically effective amount of an antidepressant, a benzodiazepine, a non-benzodiazepine hypnotic, stimulant medication, typical or atypical antipsychotic medication, NMDA antagonist, mood stabilize, anticonvulsant, buspirone, droperidol, or a prodrug or active metabolite of any of these medications, or a combination thereof.

22. The method of claim 12, wherein selecting a subject with a mood disorder or perceptual disturbance; comprises a selecting a subject with a mood disorder or perceptual disturbance in the absence of an underlying physical disorder.

23. The method of claim 12, wherein administering a therapeutically effective amount of tizanidine comprises administering a therapeutically effective amount of tizanidine in the absence of a stimulatory agent.

24. A method of treating a sleep disorder or an anxiety disorder, comprising:
selecting a subject with a sleep disturbance or an anxiety disorder; and
administering a therapeutically effective amount of tizanidine in the absence of a stimulatory agent, thereby reducing or inhibiting a symptom of the sleep disorder or anxiety disorder.

25. The method of claim 24, wherein selecting a subject with a sleep disorder or anxiety disorder comprises selecting a subject with a sleep disorder or an anxiety disorder in the absence of an underlying physical disorder.
